(19) United States
(12) Patent Application Publication
(10) Pub. No.: US 2021/0315956 A1
(43) Pub. Date: Oct. 14, 2021

(54) Therapy for the Coronavirus, COVID-19 Pandemic

(71) Applicant: Anthony Okoye Chukwudi Chibuzor, East Orange, NJ (US)

(72) Inventor: Anthony Okoye Chukwudi Chibuzor, East Orange, NJ (US)

(21) Appl. No.: 16/844,311

(22) Filed: Apr. 9, 2020

(51) Int. Cl. A61K 36/47; A61K 31/505; A61K 31/47; A61K 31/635; A61K 31/592; A61K 31/375; A61K 31/616; A61K 31/714; A61K 31/593; A61K 31/455; A61H 33/04; A61K 33/06; A61K 36/82; A61K 35/644; A61K 35/20; A61K 36/73; A61K 36/752

(52) U.S. Cl. CPC A61K 36/47; A61H 2033/048; A61K 31/47; A61K 31/635; A61K 31/592; A61K 31/375; A61K 31/616; A61K 31/714; A61K 31/593; A61K 31/455; A61H 33/04; A61K 33/06; A61K 36/82; A61K 35/644; A61K 35/20; A61K 36/73; A61K 36/752; A61K 31/505

(57) ABSTRACT

The present invention provides methods of treating a coronavirus, COVID-19 disease. COVID-19 disease is caused by the coronavirus, SARS-COV-2.

Treatment of the SARS-COV-2 infection could be achieved by administering a single element or a combination of elements in the protocol of therapies comprising of
1) A laxative
2) A Nutritious Meal followed by a cup of green tea, raw honey, lemon. milk and a dessert of pear d'anjou and strawberries
3) Administering drugs and vitamins. These drugs could be anti-parasitic, antiviral, antibacterial and/or immunomodulatory.
4) Providing for the subject infected with the SARS-COV-2 to enjoy the treat of a plain water hot bathtub bath. Some Epsom Salt should be added to the plain water hot bathtub bath. The subject should be encouraged to spend at least 30 minutes in the hot bathtub bath.
5) A Physical Exercise session.
   6 Claims, 0 Drawing Sheets.

### BACKGROUND

### The COVID-19 Disease: An Overview

COVID-19 is the code name given by World Health Organization (WHO) to an infectious disease outbreak in Wuhan, Hubei province, China. The outbreak occurred in December 2019. COVID-19 stands for Corona Virus 2019. The coronavirus, SARS-CoV-2, causes the COVID-19 disease [1, 2, 3, 4, 12].

SARS-CoV-2 stands for Severe Acute Respiratory Syndrome Coronavirus Type 2.

SARS-CoV-2 has hemagglutinin, a glycoprotein, with which it binds to cells. SARS-CoV-2 gains entry into a cell through the angiotensin-converting enzyme II (ACE2) receptor [2].

The symptoms of COVID-19 disease include fever, dry cough, fatigue, loss of taste or smell, nasal congestion, conjunctivitis, sore throat, headache, muscle or joint pain, different types of skin rash, nausea or vomiting, diarrhea, chills or dizziness, shortness of breath, loss of appetite, confusion, persistent pain or pressure in the chest, high temperature (above 38 degrees Celsius), irritability, sepsis, reduced consciousness, anxiety, depression, sleep disorders, alveolar damage, strokes, brain inflammation, delirium, nerve damage, breathing failure and death [2, 4, 11, 12].

ACE2 receptor is implicated as the entry port for the SARS-CoV-2 into the cell, and some of the cells expressing ACE2 receptors include:
I. Type II Aveolar cells (AT2) of the lung
II. Esophagus upper
III. Stratified epithelial cells
IV. Absorptive enterocytes from ileum and colon
V. Cholangiocytes
VI. Myocardial cells
VII. Myoctes
VIII. Vascular endothelial cells
IX. Kidney proximal tubule cells
X. Bladder urothelial cells [2, 4].

Organs having cells with a “high expression of ACE2 receptor”, presents potential candidates for infection of the SARS-CoV-2 [2, 11].

About 80% of subjects infected with the coronavirus, COVID-19 disease do not require treatment as their innate immune system is sufficient to fight off the viral infection. The level and quality of antibodies in a subject is essential in the fight against the SARS-CoV-2 infection [5]. Obviously, about 20% of those infected with COVID-19 disease may require treatment.

At the onset of the SARS-CoV-2 infection, macrophages are deployed to combat the infection. If the fight between the macrophages and the coronavirus, SARS-CoV-2 does not last long enough, and the macrophages are overwhelmed by SARS-CoV-2, T-Cells are recruited prematurely to engage in the fight; sometimes, this creates an out-of-control and severe inflammatory reactions of cytokines. This over reaction of cytokines is classified as “cytokine storm” and in some cases, it has fatal consequences.

SARS-CoV-2 possesses a weapon or tool, the hemagglutinin which is suspected in causing blood clotting in some infected subjects. It is also suspected that SARS-CoV-2 degrades collagen in infected subjects. Collagen is vital for gene regulation, cellular signals, cell differentiation and metabolism. The gradual degradation of collagen by SARS-CoV-2 manifests as symptoms of COVID-19 disease. Further research and clinical trials may be needed to determine and confirm these suspicions.

### Classification of Coronavirus, Covid-19 Patients

I. Mild—likely to recover from patient's immune system plus aspirin
II. Moderate—patient has fever and some symptoms of chest/lung congestion
III. Severe—patient has fever, congestion in the lung but less likely to die
IV. Critical—patient has fever, ARDS, SARS and could be on a ventilator or intubated. Patient is more likely to die.
ARDS—Acute Respiratory Distress Syndrome
SARS—Severe Acute Respiratory Disease

### DETAILED DESCRIPTION OF THE INVENTION

This invention will treat an infection of the coronavirus, COVID-19 disease by disrupting the lipid bilayer membrane of SARS-CoV-2 and disrupting the viral assembly of progenies.

The invention provides a method of treating the coronavirus, SARS-COV-2, COVID-19 disease in an infected subject comprising of some protocols and activities. Through extensive research, this invention is borne as a discovery of the potentials of some amino acids and elements that could be enhanced, exploited and administered as a therapy for the Coronavirus, COVID-19 Pandemic. Some of these amino acids and elements include—tryptophan, gluconic acid, proline and propolis.

The drugs and vitamins stated in the invention constitute the active ingredients or agents; these active ingredients combined with some activities, is propose as a treatment for an infection of the SARS-CoV-2, COVID-19 disease. This invention would cause significant distress upon the lipid bilayer membrane of SARS-CoV-2; denaturing the coronavirus, SARS-CoV-2 and potentially triggering apoptosis of the coronavirus, SARS-CoV-2. This invention is expected to boost the immune and cardiovascular systems of the subject. The invention creates a microenvironment that is hostile to the coronavirus, SARS-COV-2 but safe to the infected subject (human or non-human).

To gain the optimum benefits of this invention, the first three claims of the invention (claim 1, claim 2 and claim 3), should be administered in their entirety to a subject infected with the SARS-CoV-2, COVID-19 disease. The proposed optimum benefits notwithstanding, each claim may be administered alone or in combination with other claims of this invention or beyond this invention.

Concepts of virology and molecular biology were applied in the conception of this invention to treat an infection of the coronavirus, SARS-CoV-2 in a subject (human or non-human) [13].

### Laxative

The invention starts with administering a laxative to the subject infected with the COVID-19 disease. The laxative comprises of Castor Oil, Epsom Salt, Polyethelene glycol or Docusate sodium for enema (rectal). Administering Castor Oil, Epsom Salt, Polyethelene glycol or Docusate for enema (rectal) to a subject provides a scenario synonymous to deploying “soap” into and through the alimentary canal of the subject. The coronavirus has a fragile lipid bilayer membrane. Interactions of the laxative with the SARS-CoV-2 during cell metabolism would strip off or distress the fragile lipid bilayer of the coronavirus. The laxative would cause the bowels of the subject to be purged; causing the excretion of some level of the viral load and toxins from the infected subject.

### Sources of Amino Acids and other Elements in the Invention

A healthy meal is essential for boosting the immune system of the subject infected with COVID-19 disease. This is obvious, and this invention utilizes nutrients such as amino acid as sources of energy to boost the immune system and fight off the infection of the coronavirus, COVID-19 disease.

A nutritious meal comprising of salmon, turkey, cheese or a vegetarian delight, combined with a drink of green tea, raw honey, lemon, milk, and followed by a dessert of pear d'anjou and strawberries, will provide amino acids and elements vital to treating the SARS-CoV-2, COVID-19 disease. Some essential elements comprising of flavonols, gluconic acid, tryptophan, propolis and some measure of hydrogen peroxide formed during glycolysis constitutes nutrients and elements that would enable the disruption of the lipid bilayer of SARS-CoV-2 as well as boost the immune system of the subject.
Gluconic acid is obtained from raw honey and pear d'anjou. During glycolysis when the enzyme, glucose oxidase reacts with glucose, gluconic acid as well as hydrogen peroxide is formed [6, 14]. Some process of producing gluconic acid utilizes nitrogen and phosphorus [14]. The lipid bilayer of the coronavirus, SARS-CoV-2, has phosphorus at the ends. The E protein of SARS-CoV-2 has a negatively charge N-terminal. With the nutrients that this invention provides, during glycolysis, the phosphorus on the coronavirus presents as a reactant in cell metabolism; producing gluconic acid and hydrogen peroxide. The gluconic acid acidifies the microenvironment with a pH of about 4.5-6.0; the production of hydrogen peroxide creates a chain reaction in which hydrogen electrons from the lipid bilayer of SARS-CoV-2 are “stolen” in the chain reactions with the hydrogen peroxide radicals to form water. Such a reaction termed lipid peroxidation would denature the SARS-CoV-2. This is of vital importance as the reaction disrupts both (i) the fragile lipid bilayer of SARS-CoV-2 and (ii) the viral assembly of progenies. This will induce apoptosis of SARS-CoV-2.
Tryptophan is an amino acid that could be obtained from a nutritious meal comprising of salmon, turkey, chicken and milk. Tryptophan is vital for nitrogen balance and the creation of niacin. Niacin is vital in the production of serotonin, a neurotransmitter. Additional health benefits include the nourishing of the circadian rhythm; leading to balanced sleep, relief from depression and better pain management.
Proline is an amino acid that is obtained from raw honey [6]. Proline nourishes the muscles of the heart, joints and tendons.
Propolis is another element that offers immense health benefits such as nourishing and providing a balanced concentration of collagen. Propolis is obtained from raw honey.

### Drugs (the Active Agents) for Treating the COVID-19 Pandemic

The drugs pyrimethamine offer benefits to infected subjects who are immunocompromised. Sulfadoxine is lipid soluble and penetrates the lungs in significant concentration.
Montelukast sodium. Motelukast sodium is a leukotriene receptor antagonist that blocks the LTD4 receptor; offering relief to a congested lung by decreasing the inflammation of the lungs and causing the relaxation of smooth muscle.
Rosuvastatin is used to lower the risk of stroke and heart attack given that it works to reduce high levels of cholesterol and triglyceride levels.
Vitamins C, Vitamin B and Vitamin D supplements. Vitamin D regulates the concentration of calcium in the blood while Vitamin C is for the growth and repair of tissues. Vitamin B nourishes the energy levels, activities of the brain and general metabolism of cells.

### Hot Bathtub Bath

A hot bathtub bath saturated with Epsom salt and some body wash or gel should be included in the treatment regimen of the subject infected with SARS-CoV-2, COVID-19 disease. The subject should submerge in the bathtub. While submerged, the subject should have their eyes open. This would expose the conjunctiva to the benefits of the hot bathtub bath. The subject should spend at least 30 minutes in the hot bathtub bath. A person skilled in Epigenetics understands how this activity assists in toning and reducing the risk of inflammation in a subject.

The skin is the largest organ of the body and ACE2 receptors are extensively expressed on the skin. The hot bathtub bath offers the health benefits comprising of:
An assistance in the proper functioning of the cardiovascular system
The relief of congested lungs through the opening of alveoli
The cerebrospinal fluid is heated, soothed and energized; resulting in a lowered risk of any incidence of syringomyelia
An improved action potentials and a balanced composition of trace elements in the brain which will result in a reduced incidence of intracranial pressure (ICP). The incidence of ICP is termed “brain fog” or delirium and presents as one of several neurological symptoms of the COVID-19 disease [7, 8, 9, 11, 15]. This issue with ICP lingers on with some of the patients who have recovered from the COVID-19 disease. These patients are termed “long haulers”.
Reducing the risk of inflammation throughout the neurological and biochemical systems of the subject infected with the COVID-19 disease.

### Physical Exercise

Physical exercise sessions would assist in improving the flow of fluids in the circulatory system as well as assist in muscle, joints and tendons development and restoration. Some of the physical exercises recommended for the subject infected with the COVID-19 disease comprises of:
Undertake a significant distance walk
Ride an exercise bike
Exercise on a treadmill
Swim vigorously or partake in some vigorous exercise.

### SUMMARY

A person skilled in the art of clinical health services understands that this invention offers treatment of the SARS-CoV-2, COVID-19 Pandemic to the outpatient community, primarily. Millions of subjects are infected with the COVID-19 disease and the hospitals are overwhelmed with COVID-19 cases. This invention would assist tremendously in treating subjects infected with the Coronavirus, SARS-CoV-2 in an outpatient as well as inpatient setting.

Subjects infected with the COVID-19 disease fall into one of four categories—Mild, Moderate, Severe or Critical. The severity of the disease increases from Mild →Moderate →Severe →Critical. As the severity of the disease increases in a patient, the patient degenerates and some hundreds of thousands of patients have died from the SARS-CoV-2 infection, unfortunately. This invention offers the potentials for outpatient treatment of subjects (persons) who tested positive for the infection of SARS-CoV-2.

Subjects infected with COVID-19 disease that are at the early stages of the disease progression (Mild to Moderate stages), could self-manage their recovery from the COVID-19 disease in an outpatient setting using this invention as the therapy for coronavirus, COVID-19 disease. This invention would treat the COVID-19 disease for infected subjects in the Severe to Critical categories, also.

This invention would also treat the cases of patients who have recovered from an infection of the COVID-19 disease but are experiencing the issues of “brain fog”. Subjects in this category are termed “long haulers”. This invention would provide rehabilitation and restoration to a “new-normal-health” for these subjects who have recovered from an infection of the COVID-19 disease but yet burdened by the “long hauler” syndrome.

The elements within a claim may be administered entirely, partially or in combination thereof as the condition of the infected subject may permit.

The optimum potentials of this invention as a therapy for COVID-19 disease would be harnessed for the full benefit of an infected patient when claims 1, 2 and 3 are administered to a subject, completely.

### REFERENCES/BIBLIOGRAPHY

1. Clinical characteristics of COVID-19 patients in three consecutive generations of spread in Zhejiang, China. Y. Yao, W. Chen, X. Wu, et. al. s.l.: European Society of Clinical Microbiology and Infectious Diseases. https://doi.org/10.1016/i.cmi.2020.06.018.

2. High expression of ACE2 eceptor of 2019-nCov on the epithelial cells of oral mucosa. Hao Xu, Liang zhong, Jiaxin Deng, iakuan Peng, et. al. s.l.: International Journal of Oral Science, Feb. 24, 2020, International Journal of Oral Science (IJOS), Vol. (2020)12:8. http://doi.org/10.1038/s41368-020-0074-x.

3. Epidemiology and clinical characteristics of 99 cases of 2019 novel coronavirus pneumonia in Wuhan, China: a descriptive study. Nanshan Chen, Min Zhou, Xuan Dong, et. al. www.thelancet.com Vol 395 Feb. 15, 2020, Wuhan, China: The Lancet, 2020, Vol. 395. https://doi.org/10.1016/S0140-6736(20)30211-7.

4. Clinical course and risk factors for mortality of adult inpatients with COVID-19 in Wuhan, China: a retrospective cohort study. Fei Zhou, Ting Yu, Ronghui Du, Gouhui Fan, Ying Liu, et. al. s.l.: The Lancet, 2020, Vol. 395. https://doi.org/1.1016/S0140-6736(20)30566-3.

5. Ed Yong. Immunology Is Where Intuition Goes to Die. www.theatlantic.com. [Online] Aug. 5, 2020. [Cited: Aug. 9, 2020.] www.theatlantic.com/health/archive/2020/08/covid19-immunity-is-the-pandemics-central-mystery/614956/.

6. Bruna Costa Ferreira da Cruz, Ludmilla Ronqui, et. al. Health Benefits of Honey. www.intechopen.com. [Online] Jul. 23, 2019. [Cited: Sep. 13, 2020.] www.intechopen.com/books/honey-analysis-new-advances-and-challenges/health-benefits-of-honey. DOI: 10.5772/intechopen.88211.

7. Cerebrospinal fluid dynamics and intracranial pressure elevation in neurological diseases. Steven William Bothwell, Damir Janigro and Adjanie Patabendige. s.l.: BioMedCentral (BMC), 2019, Vol. (2019) 16:9. https://doi.org/10.1186/s12987-019-0129-6.

8. Research into the Physiology of Cerebrospinal Fluid Reaches a New Horizon: Intimate Exchange between Cerebrospinal Fluid and Interstitial Fluid May Contribute to Maintenance of Homeostasis in the Central Nervous System. Mitsunori Matsumae, Osamu Sato, Akihiro Hirayama, et. al. Tokyo: Neurol Med Chir, 2016, Vol. 56. doi: 10.2176/nmc.ra.2016-0020.

9. Nimodipine and Its Use in Cerebrovascular Disease: Evidence from Recent Preclinical and Controlled Clinical Studies. Daniele Tomassoni, Alessia Lanari, Giorgio Silvestrelli, et. al. 8, s.l.: Clinical and Experimental Hypertension, 2008, Vol. 30. http://dx.doi.org/10.1080/10641960802580232.

10. Molecular Epidemiology of Human Coronavirus OC43 Reveals Evolution of Different Genotypes over Time and Recent Emergence of a Novel Genotype due to Natural Recombination. Susanna K. P. Lau, Paul Lee, Alan K. L. Tsang, Cyril C. Y. P. Yip, et. al. 21, s.l.: Journal of Virology, November 2011, Vol. 85. doi:10.1128/JVI.05512-11.

11. Sutherland, Stephani. What We Know So Far about How COVID Affects the Nervous System. Scientific American. Oct. 22, 2020, www.scientificamerican.com/article/what-we-know-so-far-about-how-covid-affects-the-nervous-system.

12. COVID-19. World Health Organization. [Online] 2020. who.int/emergencies/diseases/novel-coronavirus-2019.

13. S. J. Flint, L. W. Enquist, V. R. Racaniello and A. M. Skalka. Principles of Virology. s.l.: American Society for Microbiology, 2009. ISBN 978-1-55581-479-3.

14. Gluconic Acid: Properties, Applications and Microbial Production. Sumitra Ramachandran, Pierre Fontanille, Ashok Pandey and Christian Larroche. 2, s.l.: Biotechnol, 2006, Vol. 44. ISSN 1330-9862 (FTB-1651).

15. The Role of Lipid Metabolism in COVID-19 Virus Infection and as a Drug Target. Mohamed Abu-Farha, Thangavel Alphonse Thanaraj, et. al. s.l.: International Journal of Molecular Sciences, 2020, Vol. 21. doi:10.3390/ijms21103544.

1. Administering some laxative comprising of (a) the Castor Oil (Ricinus Oil), (b) Epsom Salt, or (c) Polyethelene glycol mixed in a cup of green tea, raw honey, lemon and milk followed by a pear d'anjou and some strawberries. Those skilled in the art of clinical health services understand that when laxatives are administered, a lapse of time is exercised in wait.

2. Administering some drugs (the active agents) comprising of:
Pyrimethamine
Sulfadoxine
Rosuvastatin calcium
A leukotriene receptor antagonist such as Montelukast Sodium
Acetylsalicylic acid
Vitamin C
Vitamin D (preferably, vitamin D₂ or vitamin D₃)
Vitamin B (preferably, vitamin B₁₂ or vitamin B₃)

3. Providing a treat of a hot bathtub bath saturated with Epsom Salt for the subject infected with the coronavirus, SARS-COV-2, COVID-19 disease to submerge into. The bath may be scented with some aromatic scents to entice the subject to partake in the exercise. The subject should spend about 30 minutes in the hot bathtub bath.

4. A treatment of the coronavirus, COVID-19 disease wherein claim 1 is combined with claim 2 and combined with claim 3.

5. A treatment of coronavirus, COVID-19 disease wherein claim 1 is combined with claim 2.

6. A treatment of coronavirus, COVID-19 disease wherein claim 2 is combined with claim 3.
